(19) United States
(12) Patent Application Publication
(10) Pub. No.: US 2021/0337917 A1
(43) Pub. Date: Nov. 4, 2021

(54) SHOE SOLES THAT YIELD WEAR PATTERN DATA

(71) Applicant: Jinwoo Jang, Glen Ellyn, IL (US)

(72) Inventor: Jinwoo Jang, Glen Ellyn, IL (US)

(21) Appl. No.: 17/246,720

(22) Filed: May 3, 2021

Related U.S. Application Data
(60) Provisional application No. 63/019,162, filed on May 1, 2020.

(51) Int. Cl. A43B 1/00; A43B 13/12; A43B 13/14

(52) U.S. Cl. CPC A43B 1/0027; A43B 13/14; A43B 13/12

(57) ABSTRACT

The present disclosure provides shoe soles shoe soles configured to provide data about a subject's running mechanics, shoes incorporating same, and methods of making and using same.

### PRIORITY CLAIM

This application claims priority to U.S. Provisional Patent Application Ser. No. 63/019,162, filed May 1, 2020, the entire contents of which are incorporated herein by reference and relied on.

### FIELD

The present disclosure provides shoe soles configured to provide data about a subject's running mechanics, shoes incorporating same, and methods of making and using same.

### BACKGROUND

Existing methods of analyzing a walker's gait or a runner's gait typically include measuring static pressure levels associated with the subject's nonmoving posture, or analyzing qualitative features of the gait while the subject walks or runs for a few minutes on a treadmill, usually based on video obtained from a single angle (e.g., more or less a 2-dimensional perspective). Neither method provides accurate data about the subject's gait over long periods of time.

A need persists for technologies that provide convenient and accurate information about a subject's walking or running mechanics over long periods of use.

### SUMMARY

The present disclosure provides shoe soles (e.g., sole units) configured to provide data about a subject's running mechanics, shoes incorporating same, and methods of making and using same.

In some embodiments, the present disclosure provides a multilayer shoe sole for analyzing a subject's running gait, the multilayer shoe sole comprising: a base layer configured to be attached to a shoe upper; and a first indicator layer including a first indicator color disposed adjacent to the base layer; a second indicator layer including a second indicator color and disposed adjacent to the first indicator layer; and a third indicator layer including a third indicator color and disposed adjacent to the second indicator layer opposite the first indicator layer, wherein each of the first indicator layer, the second indicator layer, and the third indicator has substantially the same thickness, and wherein the first indicator color is substantially different than a color associated with the base layer.

In other embodiments, the present disclosure provides a shoe comprising a multilayer shoe sole of any one preceding claim.

In still other embodiments, the present disclosure provides a method of analyzing a running gait of a subject, the method comprising: providing a shoe as disclosed herein to the subject; capturing, after a period of time wherein the subject wears the shoe, an image of the multilayer shoe sole; and analyzing the captured image to determine at least one characteristic of the running gait of the subject.

In still other embodiments, the present disclosure provides a method of making a compensating shoe sole for a subject, the method comprising: providing a shoe as disclosed herein to the subject; capturing, after a period of time wherein the subject wears the shoe, an image of the multilayer shoe sole; analyzing the captured image to identify at least one area of relatively higher wear associated with the multilayer shoe sole; and forming a compensating shoe sole for the subject comprising at least one high density region associated with the at least one area of relatively higher wear of the multilayer shoe sole, wherein the at least one high density region has a relatively higher hardness than a second region of the compensating shoe sole that is not associated with an area of relatively higher wear of the multilayer shoe sole.

These and other embodiments are described in greater detail below with respect to the accompanying figures.

### BRIEF DESCRIPTION OF THE FIGURES

FIG. 1 shows a side plan exploded view of a shoe including a multilayer shoe sole consistent with one embodiment of the present disclosure.

FIG. 2A shows a bottom plan view of a multilayer shoe sole consistent with one embodiment of the present disclosure.

FIG. 2B shows an enlarged view of the embodiment of FIG. 2A.

FIG. 3A shows a side plan view of a shoe including a multilayer shoe sole consistent with one embodiment of the present disclosure.

FIG. 3B shows an enlarged view of the embodiment of FIG. 3A.

FIG. 4A shows a bottom plan view of a shoe including a multilayer shoe sole consistent with one embodiment of the present disclosure.

FIG. 4B shows side plan view of the embodiment of FIG. 4A.

FIG. 5A shows a bottom plan view of a shoe including a multilayer shoe sole consistent with one embodiment of the present disclosure.

FIG. 5B shows side plan view of the embodiment of FIG. 5A.

FIG. 6A shows a bottom plan view of a shoe including a multilayer shoe sole consistent with one embodiment of the present disclosure.

FIG. 6B shows side plan view of the embodiment of FIG. 6A.

FIG. 7A shows a bottom plan view of a shoe including a multilayer shoe sole consistent with one embodiment of the present disclosure.

FIG. 7B shows side plan view of the embodiment of FIG. 7A.

FIG. 8 shows a side plan view of a portion of a shoe including a multilayer shoe sole consistent with one embodiment of the present disclosure.

FIG. 9 shows a side plan view of a portion of a shoe including a multilayer shoe sole consistent with one embodiment of the present disclosure.

FIG. 10 shows a side plan view of a portion of a shoe including a multilayer shoe sole consistent with one embodiment of the present disclosure.

FIG. 11 shows a side plan view of a portion of a shoe including a multilayer shoe sole consistent with one embodiment of the present disclosure.

FIG. 12A shows a bottom plan view of a shoe including a wear pattern in a multilayer shoe sole consistent with one embodiment of the present disclosure.

FIG. 12B shows an enlargement of the embodiment of FIG. 12A.

FIG. 12C shows side plan view of the embodiment of FIG. 12A.

FIG. 12D shows an enlargement of the view of FIG. 12C.

FIG. 13 shows a bottom plan view of a compensating insole according to one embodiment of the present disclosure.

FIG. 14A shows a bottom plan view of a shoe including a compensating shoe sole consistent with one embodiment of the present disclosure.

FIG. 14B shows a side plan view of the shoe of FIG. 14B.

FIG. 15 shows a flowchart schematically representing a business method consistent with one embodiment of the present disclosure.

The figures depict various embodiments of this disclosure for purposes of illustration only. One skilled in the art will readily recognize from the following discussion that alternative embodiments of the structures and methods illustrated herein may be employed without departing from the principles of embodiments described herein.

### DETAILED DESCRIPTION

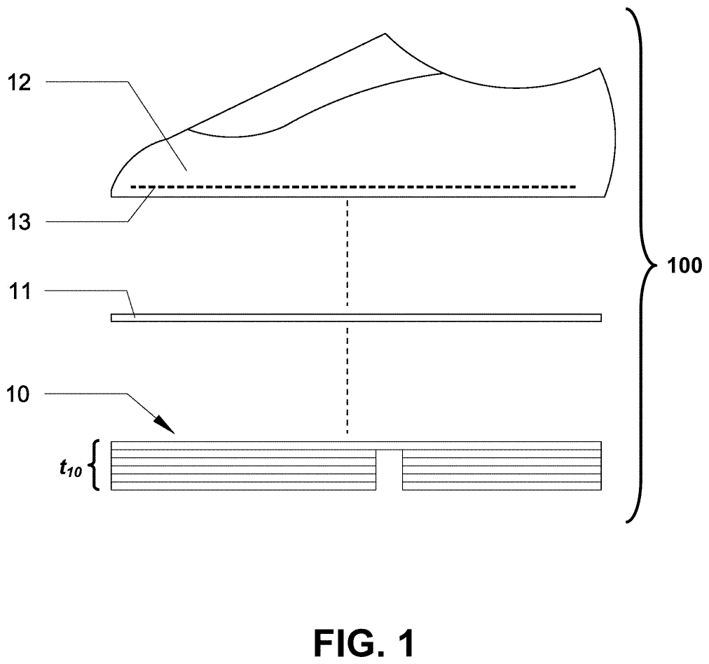

Referring generally to FIGS. 1-15, the present disclosure provides shoe soles configured to provide information about the wearer's (“subject's”) gait (e.g., walking gait or running gait), shoes incorporating same, and methods of making and using same.

Multilayer Shoe Soles and Shoes Incorporating Same

In general, shoe soles 10 consistent with the present disclosure include a plurality of indicator layers 14-19 disposed in a stacked formation. In some embodiments, each indicator layer 14-19 has a visually distinct pattern and/or color compared to the immediately adjacent indicator layer(s). In some embodiments, each indicator layer 14-19 has a visually distinct pattern and/or color compared to all other indicator layers 14-19. In some embodiments, each indicator layer 14-19 has a different color from each other layer. For example and without limitation, in embodiments consistent with that shown specifically in FIG. 3B, the base indicator layer 19 may be black, while the first indicator 18 may be red, the second indicator layer 17 may be green, the third indicator layer 16 may be yellow, the fourth indicator layer 15 may be blue, and the fifth indicator layer 14 may be orange.

Generally, the multilayer shoe soles 10 include a base layer 19 disposed on one face. The base layer 19 is configured (e.g., sized and shaped) to be attached to the upper 12 of a shoe 100. In some embodiments, the base layer 19 is secured to the upper 12 by an adhesive 11. In some embodiments, the adhesive 11 comprises, consists essentially of, or consists of a temporary adhesive, a permanent adhesive, 3M Dual Lock fastener, Velcro hook and loop fastener, mechanical fasteners, or a combination of any two or more of the foregoing.

The multilayer shoe sole 10 may be made by forming all indicator layers 14-19 simultaneously (e.g., by 3D printing all indicator layers simultaneously or sequentially in continuous or contiguous succession, by co-molding, or by co-extruding). In other embodiments, the multilayer shoe sole 10 may be made by forming some or all of the indicator layers 14-19 separately, and then fusing each of the separately formed indicator layers 14-19 together.

Each indicator layer 14-19 may be formed of any suitable material. In some embodiments, an indicator layer 14-19 comprises, consists essentially of, or consists of ethylene vinyl acetate (EVA) copolymer. The EVA copolymer may be vinyl acetate modified polyethylene, thermoplastic ethylene-vinyl acetate, or ethylene-vinyl acetate rubber. In some embodiments, an indicator 14-19 comprises, consists essentially of, or consists of rubber. The rubber may be natural rubber or synthetic rubber.

Each indicator layer 14-19 has a hardness value of about Shore 10C to about Shore 75C, for example about Shore 10C, Shore 15C, Shore 20C, Shore 25C, Shore 30C, Shore 35C, Shore 40C, Shore 45C, Shore 50C, Shore 55C, Shore 60C, Shore 65C, Shore 70C, or Shore 75C. In some embodiments, each indicator layer 14-19 has a hardness value independently selected from the group consisting of: about Shore 45C, Shore 50C, Shore 55C, Shore 60C, Shore 65C, Shore 70C, or Shore 75C. In some embodiments, each indicator layer 14-19 has a hardness value sufficient to provide a shoe sole 10 able to be used by a subject for about 20 miles to about 300 miles, for example about 20 miles, about 30 miles, about 40 miles, about 50 miles, about 60 miles, about 70 miles, about 80 miles, about 90 miles, about 100 miles, about 110 miles, about 120 miles, about 130 miles, about 140 miles, about 150 miles, about 160 miles, about 170 miles, about 180 miles, about 190 miles, about 200 miles, about 210 miles, about 220 miles, about 230 miles, about 240 miles, about 250 miles, about 260 miles, about 270 miles, about 280 miles, about 290 miles, or about 300 miles.

In some embodiments, each indicator layer 14-19 has the same or substantially the same thickness t. In other embodiments, each indicator layer 14-19 has a predetermined thickness t independently selected from the group consisting of: 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, and 25 mm.

In some embodiments, the outermost layer 14 has a thickness t configured to provide the subject with an initial break-in period. In such embodiments, the initial break-in period is provided to ensure that wear patterns of exposure of the various indicator layers 14-19 are representative of the subject's walking gait or running gait cycle mechanics (kinesiology). In such embodiments, the outermost layer 14 may have a thickness t selected from the group consisting of: 3 mm, 4 mm, 5 mm, 6 mm, 7 mm, 8 mm, 9 mm, 10 mm, 11 mm, or 12 mm.

The multilayer shoe sole 10 has a total thickness t₁₀ equal to the sum of the thickness t of each individual indicator layer 14-19. In some embodiments, the multilayer shoe sole 10 has a total thickness t₁₀ of 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, or 25 mm.

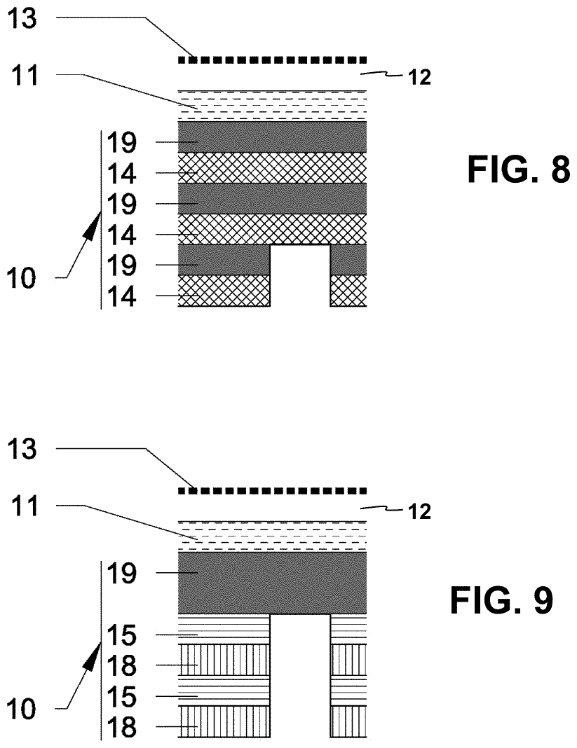

Referring now to FIG. 8, some embodiments of a multilayer shoe sole 10 include a plurality of alternating indicator layers of a first visual color and/or pattern (19) and a second visual color and/or pattern (14).

Referring now to FIG. 9, some embodiments of a multilayer shoe sole 10 include a base layer 19 having a first visual color and/or pattern, and a plurality of alternating indicator layers of a first visual color and/or pattern (15) and a second visual color and/or pattern (18).

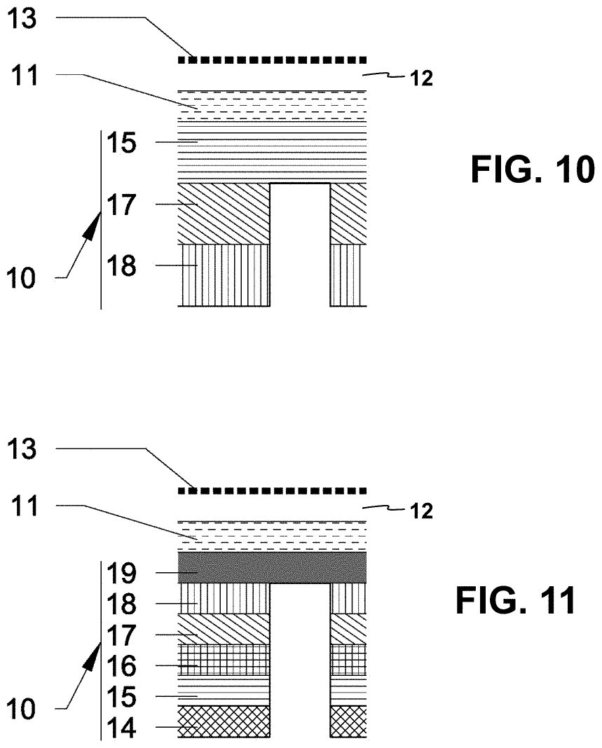

Referring now to FIG. 10, some embodiments of a multilayer shoe sole 10 do not include the base layer 19. In such embodiments, the multilayer shoe sole 10 may include a first indicator layer 15 directly adhered to the shoe upper 12 (e.g., by an adhesive layer 11) and having a first visual color and/or pattern, a second indicator layer 17 disposed adjacent to the first indicator layer 15 and having a second visual color and/or pattern, and a third indicator layer 18 disposed adjacent to the second indicator layer 17 and having a third visual color and/or pattern.

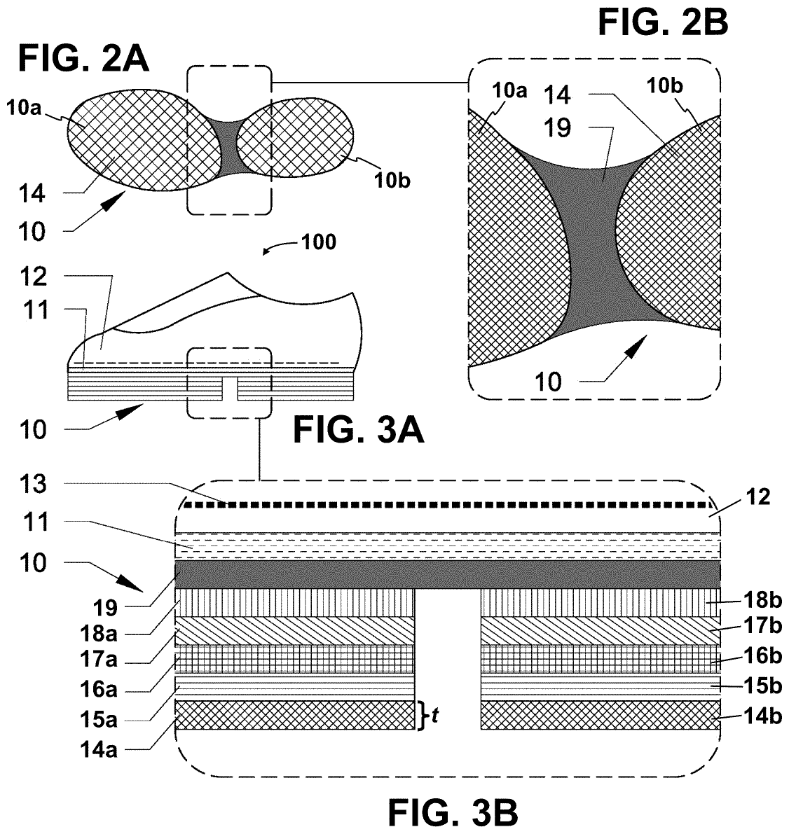

Referring now specifically to FIGS. 2A-4B, the shoe sole 10 in some embodiments includes two portions 10a-10b of the shoe sole 10. In some such embodiments, the shoe sole 10 includes a unitary base layer 19 and two portions 10a-10b each consisting of the remaining plurality of indicator layers 14a-18a and 14b-18b, respectively.

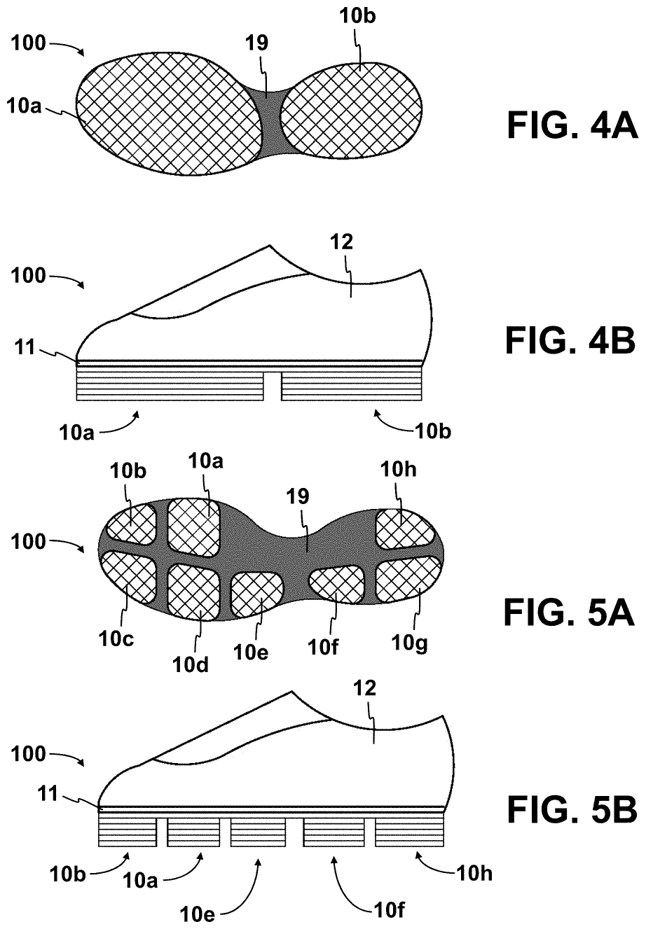

Referring now specifically to FIGS. 4A-6B, the shoe sole 10 in some embodiments includes more than two portions 10a-10h, for example 3 portions, 4 portions, 5 portions, 6 portions, 7 portions, 8 portions, 9 portions, 10 portions, or more than 10 portions. In some such embodiments, the shoe sole 10 includes a unitary base layer 19 and more than two portions 10a-10h each consisting of the remaining plurality of indicator layers 14a-18a, 14b-18b, 14c-18c, 14d-18d, 14e-18e, 14f-18f, 14g-18g, 14h-18h, etc., respectively.

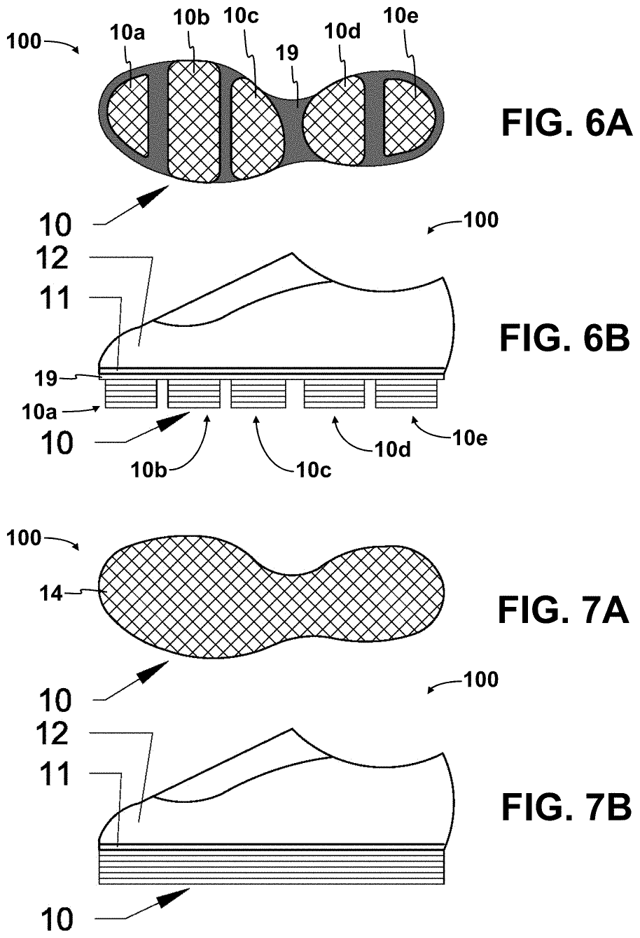

The shoe sole 10 may additionally include a medial post (not shown) disposed between or within portions of the indicator layers 10a-10h. For example and without limitation, in embodiments consistent with FIGS. 6A-6B, a medial post may be disposed between shoe sole portion 10d and shoe sole portion 10e.

In some embodiments, the shoe sole 10 includes a shank (not shown) disposed between portions of the indicator layers 10a-10h. For example and without limitation, in embodiments consistent with FIGS. 6A-6B, a shank may be disposed between shoe sole portion 10c and shoe sole portion 10d.

The present disclosure provides shoes 100 incorporating shoe soles 10 disclosed herein. In some embodiments, the shoe 100 includes an insole 13.

Methods of Analyzing Gait Characteristics

The present disclosure provides methods of analyzing (e.g., diagnosing) walking or running gait characteristics based substantially or totally on patterns of wear observable in multilayer shoe soles 10 disclosed herein.

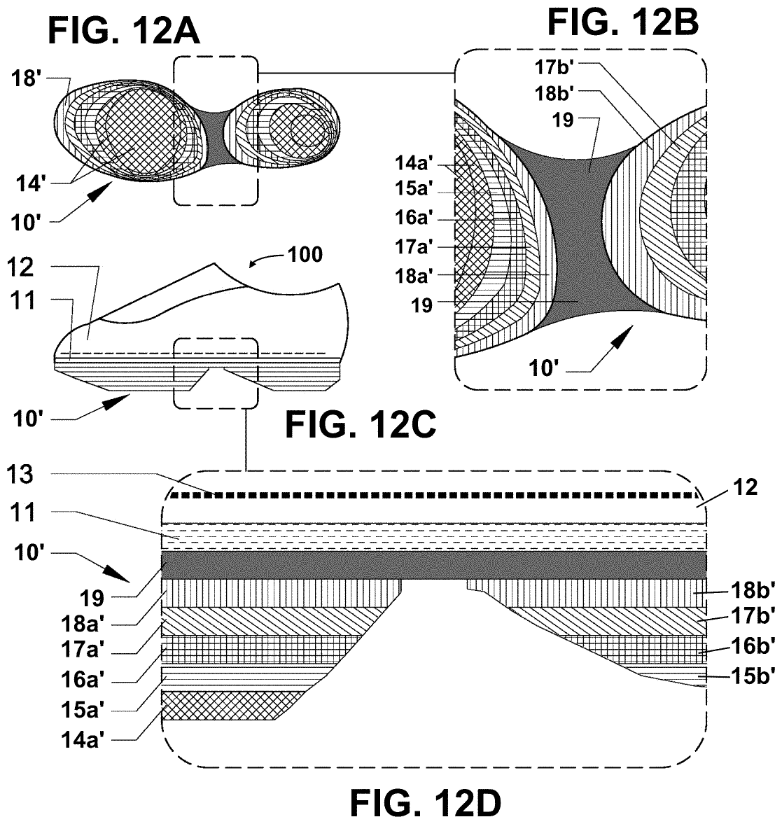

Referring now generally to FIGS. 12A-12D, after a period of use by a subject, the multilayer shoe soles 10 disclosed herein display a wear pattern 10′ associated with one or more characteristics associated with the subject's walking gait or running gait.

The wear pattern 10′ may include at least a portion 14′-18′ of all original indicator layers 14-18. In embodiments wherein the original shoe sole 10 includes a plurality of shoe sole portions 10a-10h, the wear pattern 10′ may include at least a portion 14a′-18a′, 14b′-18b′, etc. In some embodiments, the wear pattern 10′ includes a portion 14′-18′ of only some, but not all, of the original indicator layers 14-18. For example and without limitation, in some embodiments, all of an original indicator layer 14b is worn away by the subject, leaving the next layer portion 15b exposed as the new outermost layer 15b′.

When captured in two dimensions, the exposed indicator layers 14′-18′ (e.g., the wear pattern 10′) represent a topography from which wear depth from the original shoe sole 10 dimensions can be determined. This is especially true when the original indicator layers 14-18 each have a known thickness t. In general, greater wear depth corresponds to higher pressure areas between the subject's foot and the ground. Accordingly, the wear pattern 10′ consisting essentially of exposed indicator layers 14′-18′ can be used to determine physiokinetics of the subject's walking or running gait.

The method may include providing a shoe 100 including a multilayer shoe sole 10 as disclosed herein. The subject then wears the shoe 100 for a period of time, for example for a period of time to wear through at least a portion of an outermost break-in layer 14.

After the subject wears the shoe 100 for a sufficient period of time, the method may include capturing (e.g., by a camera, scanner, etc.) an image of the wear pattern 10′. The image may be transferred to a computer configured to analyze the wear pattern 10′ in the captured image. In some embodiments, the computer is configured to compare the wear pattern 10′ in the captured image to a stored image associated with an unused version of the multilayer shoe sole 10. In some embodiments, the stored image is selected by a user from a list of available stored images. In other embodiments, the computer is configured to automatically select the stored image from a database of stored images based on at least one characteristic of the multilayer shoe sole 10. For example and without limitation, the computer may be configured to automatically select the stored image based on the spatial arrangement (e.g., order) of visually distinct patterns and/or colors associated with each indicator layer 14-18. In one example embodiment, the computer may be configured to select a first stored image from the database when the wear pattern 10′ comprises a first worn layer 14′ that is red, a second worn layer 15′ that is green, and a third worn layer 16′ that is white, but select a second, different stored image from the database when the wear pattern 10′ comprises a first worn layer 14′ that is blue, a second worn layer 15′ that is yellow, and a third worn layer 16′ that is green.

The method may comprise generating, by the computer, a dynamic diagnostic image based on the comparison of the wear pattern 10′ to the stored image. The dynamic diagnostic image may show areas of relatively high pressure, areas of relatively low pressure, and/or areas of moderate pressure for the subject as determined by difference(s) between the wear pattern 10′ and the stored image. The computer may be configured to automatically generate the dynamic diagnostic image after the step of selecting the stored image occurs.

The method may further comprise comparing, by the computer, the dynamic diagnostic image to a plurality of comparative dynamic images each associated with a known characteristic of a walking gait or running gait. For example and without limitation, the plurality of comparative dynamic images may include comparative dynamic images associated with over pronation (e.g., high medial wear and/or high medial pressure), pronation (e.g., moderately high medial wear and/or moderately high medial pressure), over supination (high lateral wear and/or high lateral pressure), supination (moderately high lateral wear and/or moderately high lateral pressure), non-pronated and non-supinated (e.g., relatively even medial and lateral wear and/or pressure, or “neutral”), heavy heel striking (e.g., relatively high heel wear and/or pressure, and/or relatively low forefront wear and/or pressure), and/or heavy forefoot striking (e.g., relatively high forefront wear and/or pressure, and/or relatively low heel wear and/or pressure).

As used herein, the terms “dynamic diagnostic image” and “diagnostic image” do not necessarily mean that the images are dynamic (e.g., comprise multiple static images that, when displayed in succession, mimic a video clip). Rather, the terms “dynamic diagnostic image” and “dynamic image” are used to distinguish the present technologies over existing walking and running gait analytic methods that rely substantially or exclusively on images of a subject's foot pressure determined while the subject is standing still (i.e., is not dynamically moving). In some embodiments, the “dynamic diagnostic image” consists essentially of a single still image that displays (e.g., graphically displays) information about the subject's wear pattern 10′ resulting from the subject's use (e.g., long-term use) of a shoe 100 including a multilayer shoe sole 10 consistent with the present disclosure. Similarly, in some embodiments, the “dynamic image(s)” to which the dynamic diagnostic image is compared consists essentially of a single still image that displays (e.g., graphically displays) information about a comparative subject's wear pattern 10′ resulting from the comparative subject's use (e.g., long-term use) of a shoe 100 including a multilayer shoe sole 10 consistent with the present disclosure, wherein the comparative subject has a known abnormal walking or running gait or a known normal walking or running gait.

In some embodiments, the method further described generating a report including at least a list of one or more walking or running gait characteristics determined by the computer based on the comparison of the pressure image to the plurality of comparative pressure images. The report may further include, in some embodiments, one or more images including the comparison(s) of the pressure image to the plurality of comparative pressure images.

The method may be performed for both a left shoe 100 and a right shoe 100 associated with the same subject.

Compensating Insoles and Soles

The present disclosure also provides methods of determining a compensating insole 31 based at least in part on the wear pattern 10′ associated with an abnormal walking or running gait, for example as determined by methods disclosed herein. For example, analysis of the wear pattern 10′ (e.g., by a computer consistent with the above description) may reveal areas of high wear and/or pressure associated with the subject's walking or running gait.

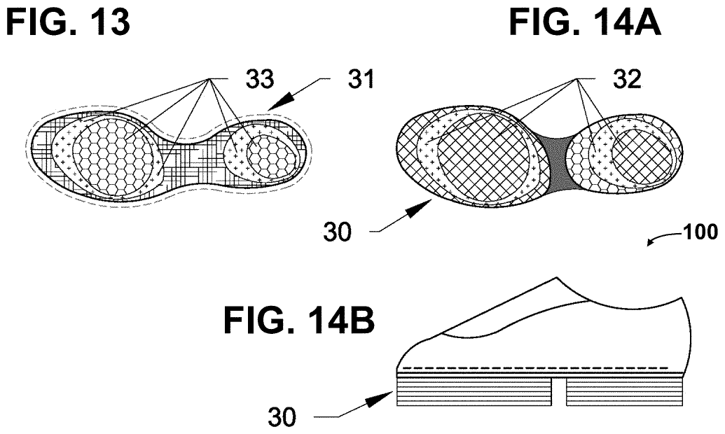

As shown representatively in FIG. 13, a compensating insole 31 may include zones 33 of relatively higher density (e.g., greater hardness) that correspond to areas of abnormally relatively higher wear in the wear pattern 10′.

The present disclosure also provides methods of determining a compensating shoe sole 30 based at least in part on the wear pattern 10′. For example, analysis of the wear pattern 10′ (e.g., by a computer consistent with the above description) may reveal areas of high wear and/or pressure associated with the subject's walking or running gait.

A method of determining a compensating shoe sole 30 may include zones 32 of relatively higher density that correspond to areas of abnormally relatively higher wear in the wear pattern 10′ (e.g., FIGS. 14A-14B).

Business Methods

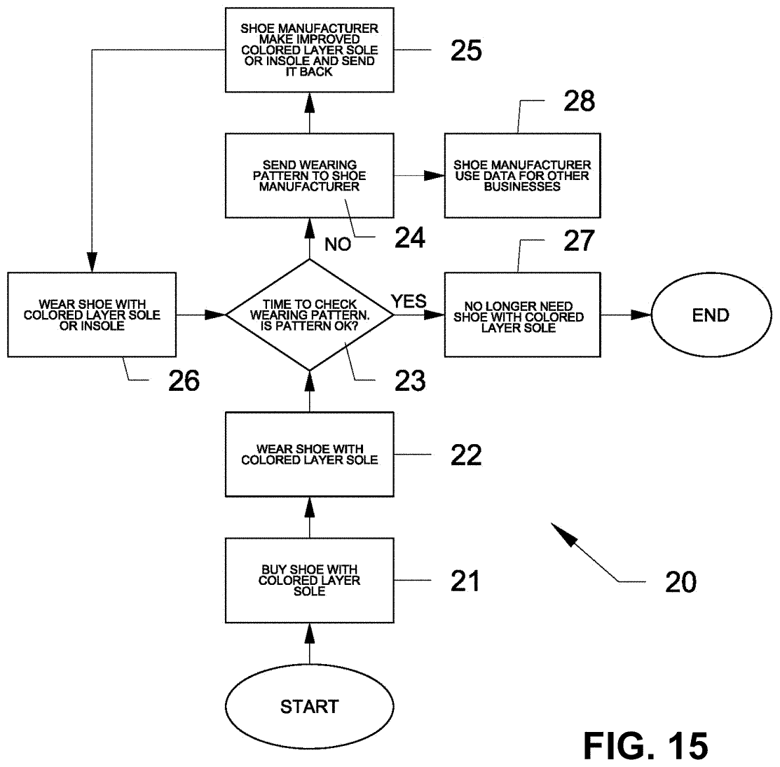

Referring now specifically to FIG. 15, the present disclosure provides methods 20 of conducting business.

In some embodiments, the method 20 includes a step 21 of providing a shoe 100 including a multilayer shoe sole 10 to a subject. A step 22 of the subject walking or running in the shoe 100 is then performed. The method 20 includes a step 23 of analyzing the multilayer shoe sole 10 to determine whether the wear pattern 10′ indicates an abnormal walking gait or running gait characteristic. If no abnormal gait characteristic is determined, the method 20 diverts via step 27 to end with a determination that the shoe 100 is no longer needed and can be replaced without a recommendation for use of a compensating insole 31 or compensating shoe sole 30. If the analysis of step 23 determines that an abnormal walking or running gait characteristic is present, the method 20 may further include a step 24 of providing the wear pattern 10′ to a shoe or insole manufacturer, who may then (step 25) manufacture a compensating insole 31 and/or a compensating shoe sole 30. The method 20 may then repeat steps 22-23 (and optionally steps 24-25) if the subject's use of the shoe(s) with compensating soles 30 and/or compensating insoles 31 generated in step 25 reveal an abnormal walking or running gait characteristic. Alternatively or in addition, the manufacturer may collect and aggregate data from a plurality of wear patterns 10′ to design mass-produced compensating insoles 31 and/or shoes with compensating shoe soles 30 addressing commonly-occurring walking gait or running gait characteristics. Such data may be made available (e.g., free of charge or for a fee) to medical professionals (e.g., podiatrists), athletic trainers, physical therapists, researchers, etc.

1. A multilayer shoe sole for analyzing a subject's running gait, the multilayer shoe sole comprising:
a base layer configured to be attached to a shoe upper; and
a first indicator layer including a first indicator color disposed adjacent to the base layer;
a second indicator layer including a second indicator color and disposed adjacent to the first indicator layer; and
a third indicator layer including a third indicator color and disposed adjacent to the second indicator layer opposite the first indicator layer,
wherein the first indicator color is substantially different than a color associated with the base layer.

2. The multilayer shoe sole of claim 1, wherein each of the first indicator layer, the second indicator layer, and the third indicator has substantially the same hardness value.

3. The multilayer shoe sole of claim 1, wherein:
the second indicator color is substantially different than the first indicator color, and
the second indicator color is substantially different than the third indicator color.

4. The multilayer shoe sole of claim 1, wherein the third indicator color is substantially different than the first indicator color.

5. The multilayer shoe sole of claim 1, wherein each of the first indicator layer, the second indicator layer, and the third indicator has substantially the same thickness.

6. The multilayer shoe sole of claim 1, wherein the third indicator layer is substantially thicker than either of the first indicator layer and the second indicator layer.

7. The multilayer shoe sole of claim 1 further comprising a fourth indicator layer including a fourth indicator color and disposed adjacent to the third indicator layer opposite the second indicator layer, wherein the fourth indicator color is substantially different than the third indicator color.

8. The multilayer shoe sole of claim 7, wherein the fourth indicator layer is substantially thicker than each of the first indicator layer, the second indicator layer, and the third indicator layer.

9. The multilayer shoe sole of claim 7 further comprising a fifth indicator layer including a fifth indicator color and disposed adjacent to the fourth indicator layer opposite the third indicator layer, wherein the fifth indicator color is substantially different than the fourth indicator color.

10. The multilayer shoe sole of claim 9, wherein the fifth indicator layer is substantially thicker than each of the first indicator layer, the second indicator layer, the third indicator layer, and the fourth indicator layer.

11. The multilayer shoe sole of claim 9 further comprising a sixth indicator layer including a sixth indicator color and disposed adjacent to the fifth indicator layer opposite the fourth indicator layer,
wherein the sixth indicator color is substantially different than the fifth indicator color.

12. The multilayer shoe sole of claim 11, wherein the sixth indicator layer is substantially thicker than each of the first indicator layer, the second indicator layer, the third indicator layer, the fourth indicator layer, and the fifth indicator layer.

13. The multilayer shoe sole of claim 1, wherein after a period of use by a subject, the multilayer shoe sole displays a wear pattern (e.g., a topographical contour pattern associated with observable boundaries between adjacent indicator layers) associated with one or more characteristics associated with a running gait of the subject.

14. The multilayer shoe sole of claim 1, wherein the outermost indicator layer disposed opposite the base layer is substantially thicker than any other indicator layer.

15. A shoe comprising a multilayer shoe sole of claim 1.

16-21. (canceled)
